(19) United States
(12) Patent Application Publication
(10) Pub. No.: US 2021/0323995 A1
(43) Pub. Date: Oct. 21, 2021

(54) ISOLITHOCHOLIC ACID OR ISOALLOLITHOCHOLIC ACID AND DEUTERATED DERIVATIVES THEREOF FOR PREVENTING AND TREATING CLOSTRIDIUM DIFFICILE-ASSOCIATED DISEASES

(71) Applicant: PHENEX PHARMACEUTICALS AG, Ludwigshafen (DE)

(72) Inventors: Christian Gege, Ehingen (DE); Manfred Birkel, Seeheim-Jugenheim (DE); Thomas Hoffman, Ketsch (DE)

(73) Assignee: PHENEX PHARMACEUTICALS AG, Ludwigshafen (DE)

(21) Appl. No.: 17/267,449

(22) PCT Filed: Aug. 8, 2019
(86) PCT No.: PCT/EP2019/071315

§ 371 Date: Feb. 9, 2021

(30) Foreign Application Priority Data
Aug. 10, 2018 (EP) 18188420.6

(51) Int. Cl. C07J 9/00; A61P 31/04

(52) U.S. Cl. CPC C07J 9/005; C07B 2200/05; A61P 31/04

(57) ABSTRACT

The present invention relates to isolithocholic acid (3ß-hydroxy-5ß-cholan-24-oic acid, iso-LCA) and isoallolithocholic acid (3ß-hydroxy-5α-cholan-24-oic acid) and their deuterated analogs for preventing or treating Clostridium difficile-associated disease in a mammalian subject.

The present invention relates to isolithocholic acid (3β-hydroxy-5β-cholan-24-oic acid, iso-LCA) and isoallolithocholic acid (3β-hydroxy-5α-cholan-24-oic acid) and their deuterated analogs for preventing or treating Clostridium difficile-associated disease in a mammalian subject.

### INTRODUCTION

Clostridium difficile (abbreviated C. difficile or C. diff.; also termed Clostridioides difficile) is an anaerobic, gram-positive spore-forming rod-shaped bacterium. Sporulation is important to the capacity of this organism to cause disease, as the spores (endospores) persist on environmental surfaces, and are resistant to a number of disinfectants and antibiotics, thereby facilitating transmission. Change of environmental conditions trigger the germination of the spores and the bacteria can proliferate. Members of the genus Clostridium are: C. perfringens, C. tetani, C. botulinium, C. sordellii and C. difficile. Clostridia are associated with diverse human diseases including tetanus, gas gangrene, botulism and pseudomembranous colitis and can be a causative agent in food poisoning.

C. diff causes Clostridium difficile-associated diseases (CDAD) or Clostridium difficile infection (CDI). Over the past decade, the number of CDI has significantly increased with hyper-virulent and drug resistant strains now becoming endemic. CDI is primarily of concern in the hospital setting and is of particular concern amongst elderly patients where mortality rates are particularly high. Of particular concern is the emergence of new endemic strains. A particularly pertinent example is the hyper-virulent BI/NAP1/027 (also known as ribotype 027) strain which shows increased toxin A and B production as well as the production of additional novel binary toxins.

CDI is a serious issue in the Western World with estimates of up to 700.000 cases of CDI per year in the US alone. The US Center for Disease Control and Prevention report that CDI is responsible for 14.000 deaths per annum in the US and has designated C. diff. as one of three pathogens that poses an immediate public health threat and requires urgent and aggressive action.

C. diff. is a commensal enteric bacterium, the levels of which are kept in check by the normal gut flora. Disruption of indigenous bacterial flora in the intestinal tract by antimicrobial therapy (or, occasionally, by chemotherapy) is a critical element in the pathogenesis of infection. With the understanding that this infection is a complication of antimicrobial therapy, an important therapeutic intervention is discontinuation of the offending drug when possible. Exposure to C. diff. may lead to asymptomatic colonization or infection. Infection is associated with a wide spectrum of clinical manifestations from mild diarrhea through to death.

The current standard of care CDI treatments are the broad spectrum antibiotics, vancomycin and metronidazole. While effective at reducing levels of C. diff., these antibiotics also cause significant collateral damage to the gut flora because of their broad spectrum activity and leave patients vulnerable to disease recurrence, the primary clinical issue. Each additional episode of the disease is associated with greater disease severity and higher mortality rates. It has been reported that approximately 25% of CDI patients suffer a second episode of the infection, and the risk of further recurrence rises to 65%. Recurrent disease is associated with an increased burden on the healthcare system. Although clindamycin is the major antibiotic associated with CDAD, the disease is now associated with nearly all antibiotics including members of the fluoroquinolone, cephalosporin, macrolide, β-lactam and many others classes.

Current Treatment Options for CDI

Antibiotic Therapy

The Infectious Diseases Society of America (IDSA) currently recommends Metronidazole as the therapeutic agent of choice for mild CDI and Vancomycin for severe CDI.

Newer antibiotics, which are already approved (Fidaxomicin approved by the FDA in May 2011, DIFICID®, formerly referred to as OPT-80) or which are in development (Ridinilazole), are aiming to be more effective against C. diff, but trying to spare the healthy gut microbiome. Additionally, these antibiotics are limited to the intestine with no systemic exposure due to low oral bioavailability.

Antitoxins

Bile acid sequestrants like Cholestyramine (Questran) binds toxins A and B of C. diff., but the clinical experience of different investigators has shown marked variation in results. Cholestyramine binds vancomycin and should not be used concurrently with vancomycin therapy.

Actoxumab and Beziotoxumab are fully human monoclonal antibodies which binds toxins A and B of C. diff., respectively. These antibodies are designed for the prevention of recurrence of CDI but due to the mechanism of action, reduce only the symptoms of the disease but do not eradicate the cause of the disease the CDI. Beziotoxumab (Zinplava) was approved in October 2016 by the U.S. FDA.

Vaccination

Vaccines based on the neutralization of bacterial toxins have already proven efficacy as illustrated by the decreased prevalence of disease caused by Corynebacterium diphteriae or Clostridium tetani in countries where vaccination programs include these two toxoid vaccines. Currently, two vaccine candidates against CDI are being clinically tested, Sanofi-Pasteur's toxoid ACAM-CDIFF™ composed of a mixture of formalin-inactivated toxin A and B and Intercell's recombinant fusion protein containing a part of the receptor-binding domain of toxins A and B as an anti-CDI vaccine candidate.

Gut Microbiome Modulation

Probiotics are not recommended as a single agent for the treatment of active CDI owing to limited data supporting their benefit and a potential risk for septicemia.

However, as it becomes more and more obvious that disruption of the healthy bowel flora is in general the basis for relapsing CDI, restoration of the normal colonic bacterial flora seems to be optimal for the prevention of disease recurrence. This could be e.g. achieved by fecal microbiota transplantation (FMT) which reported clinical cure rates for recurrent CDI with more than 90%.

### Prior Art

Bile Acids and Clostridium difficile

Bile Acids as Germination Drivers of C. difficile

Germination of spores of C. diff within the gastrointestinal tract of a host is critical to initiate C. diff.-associated diseases since only the vegetative form produces toxin. In general, bacterial spores germinate in a specific environment in the host, often in response to the binding of one and or more small molecules. In case of C. diff, it was first in vitro shown that different conjugates as well as unconjugated primary bile acids such as cholate, taurocholate and glycocholate are able to stimulate germination (J. A. Sorg & A. L. Sonenshein, J. Bacteriol. 2008; 180:2505).

Later experiments in mice proved in vivo that bile acids are related to the germination and disease initiation. Treatment of mice with cholestyramine, a bile salt binding resin, severely decreased the germination capacity of C. diff. spores. On the other hand, treatment of mice with antibiotics stimulated the germination capability in vivo. It was further shown in mice that this effect of antibiotics in the animal model was related to a higher proportion of primary to secondary bile acids in the stool of antibiotic treated mice (J. L. Giel et al., PlosOne 2010; 5:e8740).

These new findings directly lead to the idea that bile acids or bile acids derivatives could be therapeutically used to block spore germination in vivo and thereby the initiation of CDI disease.

It was shown that all bile acids lacking a 12α-hydroxyl group on the bile acid scaffold could be in principal used as competitive inhibitors of spore germination (competitive to all bile acids in the host with a 12α-hydroxyl moiety that drives germination).

Further structure-activity-relationship work done on the bile acid scaffold by the Sorg group exemplified that an ester moiety compared to a free carboxylic acid would be an even more preferred structure since this was associated with significant lower inhibitor constants (Ki).

Furthermore, it was postulated that effective inhibitors need to resist uptake by the colonic epithelium and to resist 7-dehydroxylation by the colonic gut flora. Therefore the acetylation of i.e. the 7-OH position of the bile acid scaffold was proposed to be preferred (J. A. Sorg & A. L. Sonenshein, J. Bacteriol. 2008; 180:2505 as well as WO2010/062369 and WO2015/076788). Noteworthy to mention is the fact, that although free bile acids (including lithocholic acid (LCA); see claim 9) are claimed in WO2010/062369 for the treatment of CDI, LCA was never tested and a 3-O-substituted LCA derivative (considered as closest example towards our invention) showed no effect on C. diff, spore germination (see Table 3: 5β-cholanic acid 3α-ol acetate) in contrast to bile acid esters. In WO2010/062369 only 3α-O bile acids are described, while both 3α-O and 3β-O bile acids are claimed. Noteworthy, no deuterated bile acids are mentioned. WO2015/076788 is restricted to muricholic acid-based compounds (containing a 6-hydroxy moiety in the steroidal core).

For LCA several liabilities in the literature are reported: for example oral administration of LCA results in elevation of alanine transaminase (ALT) indicating hepatocellular injury (A. F. Hofmann, Drug Metab. Rev. 2004; 36:703; B. L. Woolbright et at, Toxicol. Lett. 2014; 228:56). We were able to confirm this described liability of ALT-elevation at even lower dose (FIG. 1a). In addition, LCA is described as Vitamin D agonist (M. Ishizawa et at, J. Lipid Res. 2008; 49:763; R. Adachi et al., J. Lipid Res. 2005; 46:46), however higher doses can lead to hypercalcemia followed by polyuria. We confirmed this Vitamin D agonism (Example 202) for LCA and showed, that iso-LCA and analogs are devoid of this Vitamin D agonism.

Bile Acids as Inhibitors of C. difficile Growth

Until 2015 the therapeutic application of bile acid derivatives for treatment of C. diff.-infections was only seen in their inhibitory potential on the germination step which usually takes place in the small intestine of a host. However a recent series of papers showed in different mouse models that secondary bile acids are even capable to prevent the outgrowth of C. diff, vegetative cells in the large intestine—an effect that was fully independent of the before reported effects of bile acids on spore germination (C. G. Buffie et al., Nature 2015; 517:205, M. J. Koenigsknecht et at, Infect, Immun. 2015; 83:934, C. M. Theriot & V. B. Young, Annu. Rev. Microbiol. 2015:69:445).

It could be shown in murine models of CDI that there is a direct correlation between the large intestinal amount of secondary bile acids (LCA and deoxycholic acid (DCA)) and the severity of CDI. It was demonstrated that a healthy microbiota that is able to generate enough secondary bile acids through the process of bile acid metabolism in the gastrointestinal tract protects the host against the outgrowth of C. diff, in the large intestine even if the host is challenged with vegetative C. diff. bacteria.

Once the natural bile acid metabolism is interrupted by antibiotic therapy, no or not enough secondary bile acids are generated which makes the host vulnerable for a C. diff disease.

Of special importance for the protection of the host seem to be the secondary bile acid producing bacteria strains, e.g. Clostridium scindens, which carry the enzyme 7α-dehydroxylase. The reconstitution of the microbiome after antibiotic challenge with the single bacterial strain Clostridium scindens was e.g. sufficient to protect against CDI in a mouse model (C. G. Buffie et al. Nature 2015; 517:205).

These findings in murine models were further confirmed by human data: A patient with recurrent C. diff. infections was treated with UDCA and remained infection-free for over 10 months (A. R. Weingarden et al, J. Clin. Gastroenterol. 2016; 50:624). Also systematic bile acid profiling in the stool of patients with first time CDI, patients with recurrent CDI and healthy controls mirrors the profiles seen in the mouse models. Secondary bile acids in stool were significantly depleted in the most severe cases of CDI whereas primary bile acids in stool were elevated in recurrent CDI (J. R. Allegretti et al., Aliment. Pharmacol. Ther. 2016; 43:1142).

The impact of various secondary bile acids (including iso-LCA) on different C. diff. strains was investigated in vitro by R. Thanissery et al. in Anaerobe 2017; 45:86. The study illustrates how C. diff. strains can have different responses when exposed to secondary bile acids in vitro. Many secondary bile acids are able to inhibit TCA mediated spore germination and outgrowth and toxin activity in a dose dependent manner, but the level of inhibition and resistance varied across all strains and ribotypes. Bile acid sensitivity and in vivo virulence of C. diff, clinical isolates using LCA and DCA in in vitro investigations was described by B. B. Lewis et al. in Anaerobe 2016; 41:23.

Finally, also the enormous clinical success of fecal microbiota transplantation (FMT) for last-line treatment of patients with several rounds of recurrent CDI could be attributed to reintroduction of bacterial strains with 7α-dehydroxylase activity.

Deuterated Bile Acids

Deuterated bile acids are described for closed analogs, e.g. obeticholic acid (WO2016/131414, CN105985396, CN106008639, WO2016/168553 or WO2019/023103 as well as by K. Gai et al. in J. Label. Compd. Radiopharm. 2018:61:799) or 3β,12α-dihydroxy-5-cholen-24-oic acid (M. Tohma et al. in J. Chromatogr. Biomed. Applic. 1987; 421:9). (so-bile acids (30-hydroxy) are described for chenodeoxycholic acid (F. Aragozzini et al., Biochem. J. 1985; 230:451), where 3-deuterated iso-CA was investigated in the mechanism of 3-hydroxy epimerisation by Clostridium perfrigens. The tritium-labeled methyl ester of iso-LCA was described by A. F. Hofmann et al. in J. Lipid Res. 1968; 9:707.

Remaining Challenges

In summary, secondary bile acids have a direct impact on vegetative C. diff cells by growth inhibition, so that they could be used theoretically to treat and/or prevent CDI or recurrent CDI as a kind of supplement for too low colonic secondary bile acid exposure due to antibiotic disturbance of the microbiota. However, several issues are attached to this:

1) A first disadvantage of a direct C. diff. therapy with either natural LCA or DCA are the toxic properties of high LCA/DCA concentrations on colon and liver tissue. E.g. high systemic LCA exposure is related i.e. to liver diseases (B. L. Woolbright et al. in Toxicol. Left. 2014; 228:56). whereas DCA is known for his proliferative effect on colon tissue and is related to the occurrence of colon tumors (Y. H. Ha et al. in J. Korean Soc. Coloproctol. 2010; 26:254). Therefore, systematic and intestinal exposure to these secondary bile acids should be rather limited.

2) A second limitation is the pharmacokinetics of secondary bile acids i.e. with limited colonic exposure due to high absorption especially by the ileal ASBT transporter. This makes it difficult to achieve sufficient exposure in colon and is directly linked to the first disadvantage—the unwanted systemic exposure of both compounds.

Based on this, we started testing of isolithocholic acid (5β-cholanic acid-3β-ol) or isoallolithocholic acid (5α-cholanic acid-30-ol) and their deuterated derivatives and we surprisingly identified the claimed compounds as being preferred over LCA In animal models of C. difficile. A head-to-head comparison of iso-LCA and LCA confirmed a better efficacy expressed as a higher surviving rate in the recurrence mouse model (Example 202; FIG. 2).

### SUMMARY OF THE INVENTION

The present invention relates to a compound according to Formula (I)

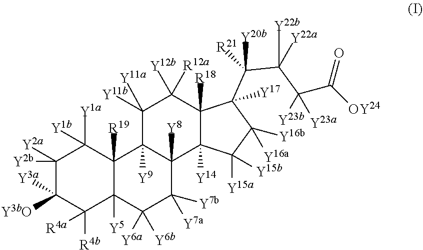

or a pharmaceutically acceptable salt, co-crystal or solvate thereof,
wherein each R¹⁸, R¹⁹ and R²¹ is independently selected from —CH₃, —CH₂D, —CHD₂ and —CD₃; and each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium, for use in preventing or treating a Clostridium difficile-associated disease in a mammalian subject.

The invention further relates to a compound represented by Formula (II)

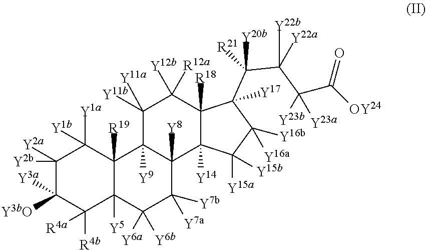

of or a pharmaceutically acceptable salt, co-crystal or solvate thereof, wherein
each R¹⁸, R¹⁹ and R²¹ is independently selected from —CH₃, —CH₂D, —CHD₂ and —CD₃; each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium; with the proviso that at least one of Y^{1a}, Y^{1b}, Y^{2a}, Y^{1b}, Y^{3a}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a} and Y^{23b} is deuterium; or at least one of R¹⁸, R¹⁹ and R²¹ is selected from —CH₂D, —CHD₂ and —CD₃.

The invention also relates to compounds according to Formula (II) for use in preventing or treating a Clostridium difficile-associated disease.

In a further aspect, the present invention relates to a method of preventing or treating a Clostridium difficile-associated disease in a mammalian subject, comprising administering to a mammalian subject having or is at risk of developing Clostridium difficile-associated disease an effective amount of a compound of Formula (I)

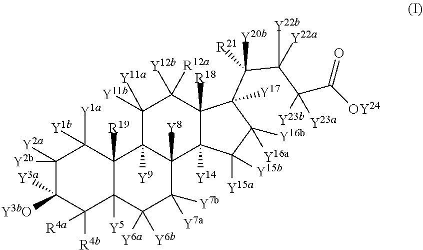

or a pharmaceutically acceptable salt, co-crystal or solvate thereof, wherein each R¹⁸, R¹⁹ and R²¹ is independently selected from —CH₃, —CH₂D, —CHD₂ and —CD₃; and each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium.

### BRIEF DESCRIPTION OF THE DRAWINGS

FIG. 1a shows the ALT plasma concentration of mice after administration of 500 mg/kg LCA for 5 days.

FIG. 1b shows the ALT plasma concentration of mice after administration of 500 mg/kg iso-LCA and deuterated iso-LCA for 5 days.

FIG. 2 shows a head-to-head comparison (survival) of iso-LCA versus LCA in a recurrence model mouse.

FIG. 3 shows a comparison of iso-LCA and deuterated iso-LCA in a recurrence model mouse.

FIG. 4 depicts the result of iso-LCA in a recurrence model hamster.

FIG. 5 depicts the result of iso-LCA in a recurrence model hamster with two different doses.

### DETAILED DESCRIPTION OF THE INVENTION

More precisely, the present invention relates to a compound according to Formula (I)

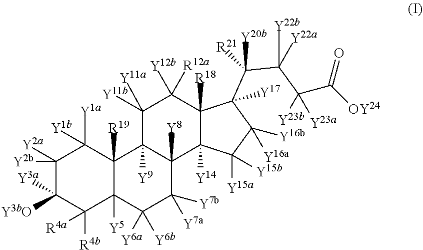

or a pharmaceutically acceptable salt, co-crystal or solvate thereof,
wherein each R¹⁸, R¹⁹ and R²¹ is independently selected from —CH₃, —CH₂D, —CHD₂ and —C₃; and each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium, for use in preventing or treating a Clostridium difficile-associated disease in a mammalian subject.

In a preferred embodiment, the compound of Formula (I) or a pharmaceutically acceptable salt, co-crystal or solvate thereof for use in preventing or treating a Clostridium difficile-associated disease in a mammalian subject is selected from

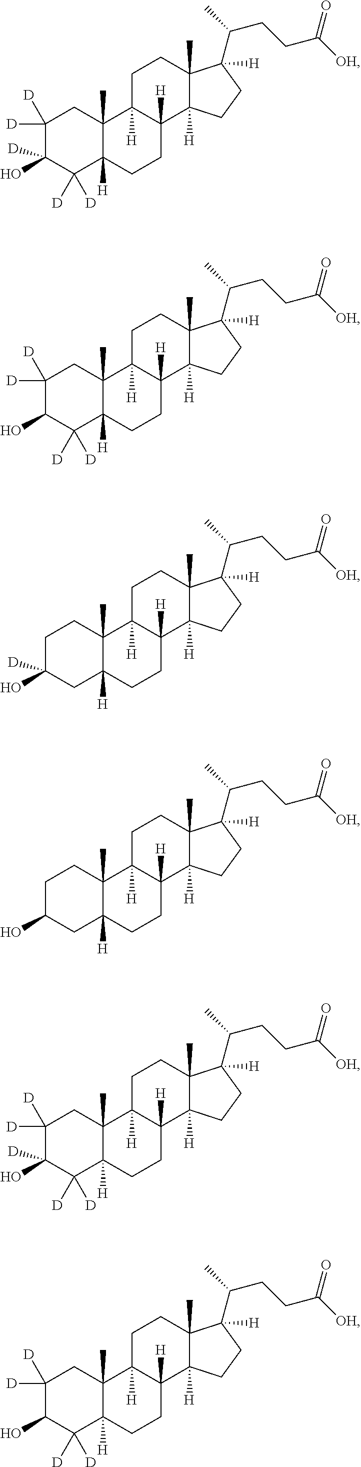

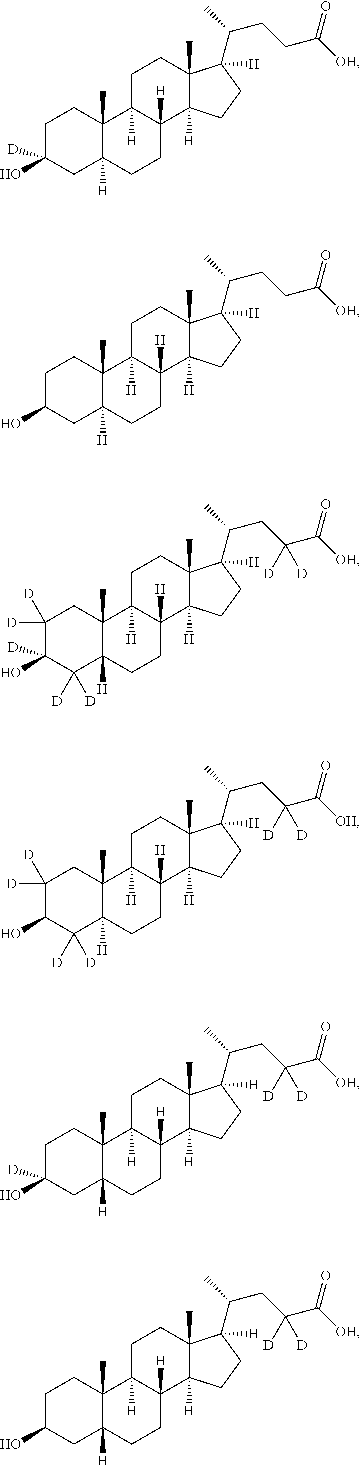

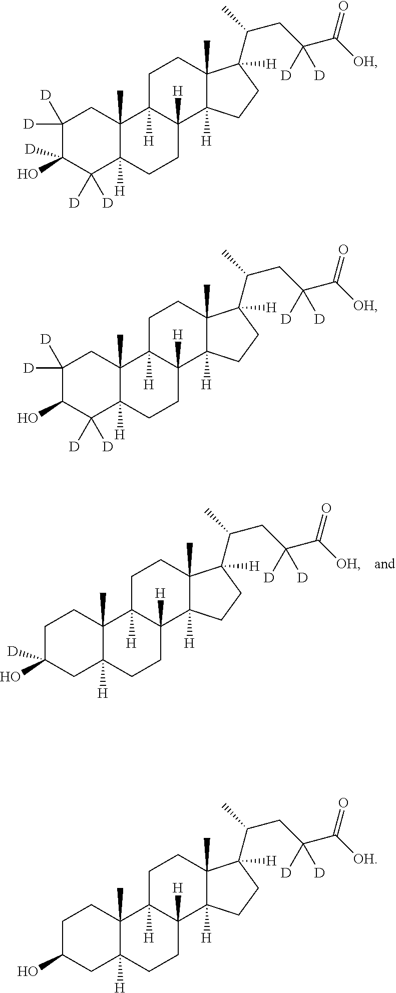

In a more preferred embodiment, the compound of Formula (I) or a pharmaceutically acceptable salt, co-crystal or solvate thereof for use in preventing or treating a Clostridium difficile-associated disease in a mammalian subject is selected from

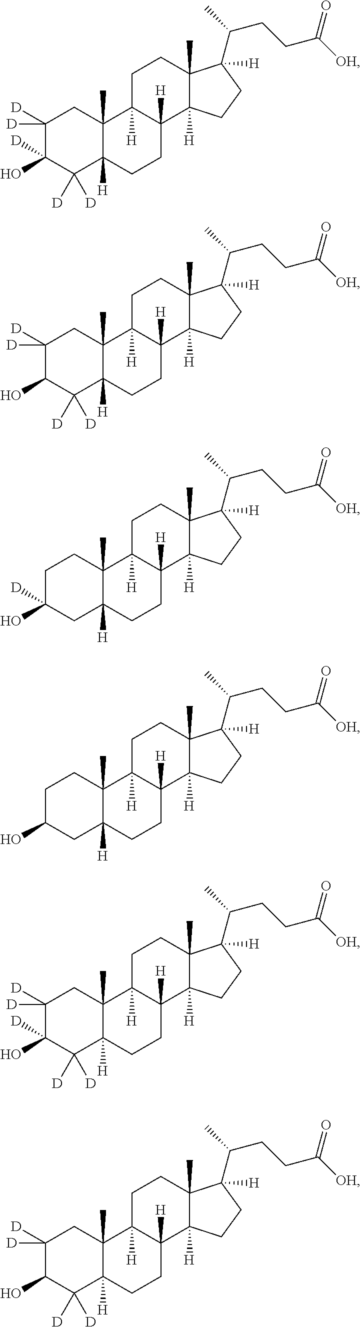

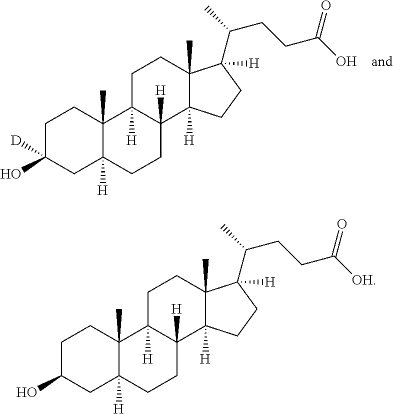

In a further preferred embodiment, Y⁵ in Formula (I) is in the beta-orientation.

In a more preferred embodiment, the compound of Formula (I) or a pharmaceutically acceptable salt, co-crystal or solvate thereof for use in preventing or treating a Clostridium difficile-associated disease in a mammalian subject is selected from

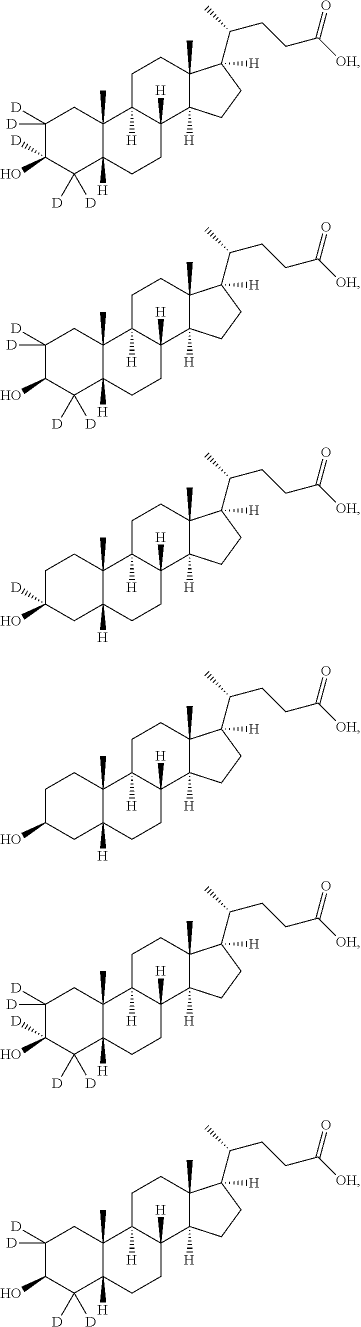

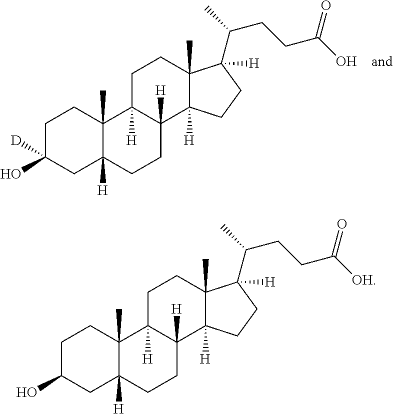

In an even more preferred embodiment, the compound of Formula (I) or a pharmaceutically acceptable salt, co-crystal or solvate thereof for use in preventing or treating a Clostridium diffcile-associated disease in a mammalian subject is selected from

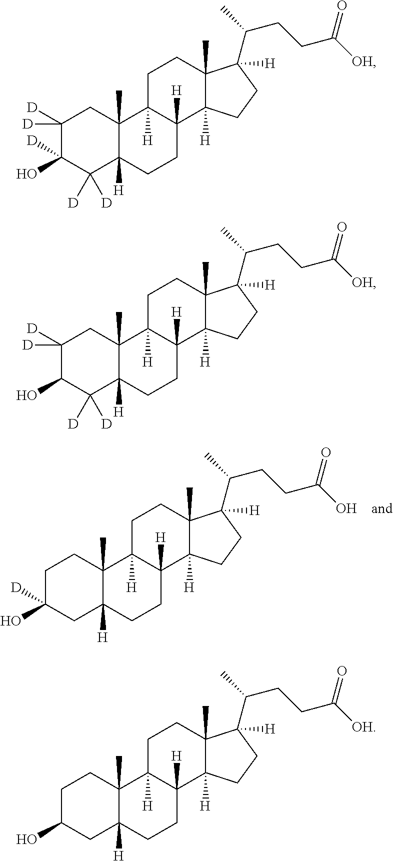

In yet another preferred embodiment, the compound of Formula (I) or a pharmaceutically acceptable salt, co-crystal or solvate thereof for use in preventing or treating a Clostridium difficile-associated disease in a mammalian subject is

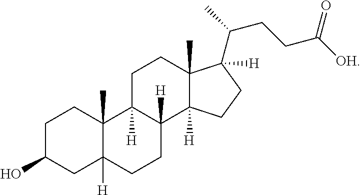

In a more preferred embodiment, the compound of Formula (I) or a pharmaceutically acceptable salt, co-crystal or solvate thereof for use in preventing or treating a Clostridium difficile-associated disease in a mammalian subject is

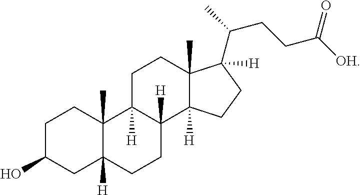

In a further preferred embodiment, the compound of Formula (I) or a pharmaceutically acceptable salt, co-crystal or solvate thereof for use in preventing or treating a Clostridium difficile-associated disease in a mammalian subject is

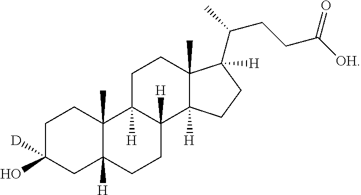

The invention further relates to a compound represented by Formula (II)

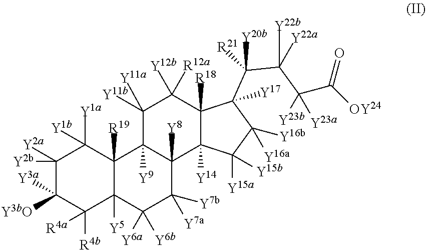

or a pharmaceutically acceptable salt, co-crystal or solvate thereof, wherein each R¹⁸, R¹⁹ and R²¹ is independently selected from —CH₃, —CH₂D, —CHD₂ and —CD₃; each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium;
with the proviso that at least one of Y^{1a}, Y^{1b}, Y^{2a}, Y^{1b}, Y^{3a}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, and Y^{23b} is deuterium; or at least one of R¹⁸, R¹⁹ and R²¹ is selected from —CH₂D, —CHD₂ and —CD₃.

In a more preferred embodiment of the compound of Formula (II) or a pharmaceutically acceptable salt, co-crystal or solvate thereof, each Y^{2a}, Y^{2b}, Y^{3a}, Y^{3b}, Y^{4a}, Y^{4b}, Y^{23a}, Y^{23b} is independently selected from hydrogen or deuterium; each R¹⁸, R¹⁹ and R²¹ is —CH₃; and each Y^{1a}, Y^{1b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b} and Y²⁴ is hydrogen, with the proviso that at least one of Y^{2a}, Y^{2b}, Y^{3a}, Y^{3b}, Y^{4a}, Y^{4b}, Y^{23a}, Y^{23b} is deuterium.

In an even more preferred embodiment of the compound of Formula (II), each Y^{2a}, Y^{2b}, Y^{3a}, Y^{3b}, Y^{4a}, Y^{4b} is independently selected from hydrogen or deuterium; each R¹⁸, R¹⁹ and R²¹ is —CH₃; and each Y^{1a}, Y^{1b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is hydrogen, with the proviso that at least one of Y^{2a}, Y^{2b}, Y^{3a}, Y^{3b}, Y^{4a}, Y^{4b}, Y^{23a}, Y^{23b} is deuterium.

In a more preferred embodiment, the compound of Formula (II) or a pharmaceutically acceptable salt, co-crystal or solvate thereof is selected from

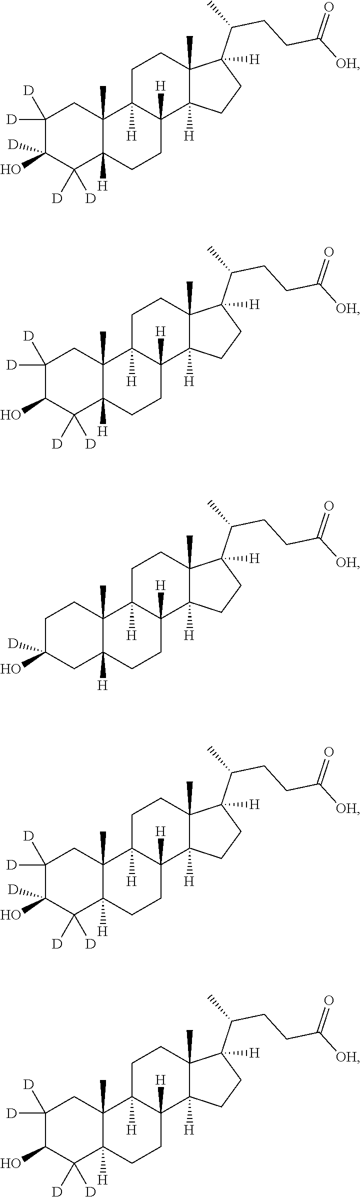

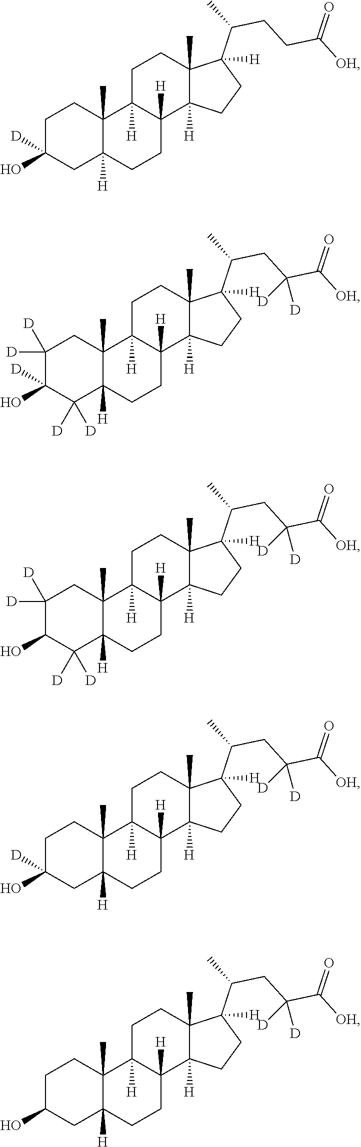

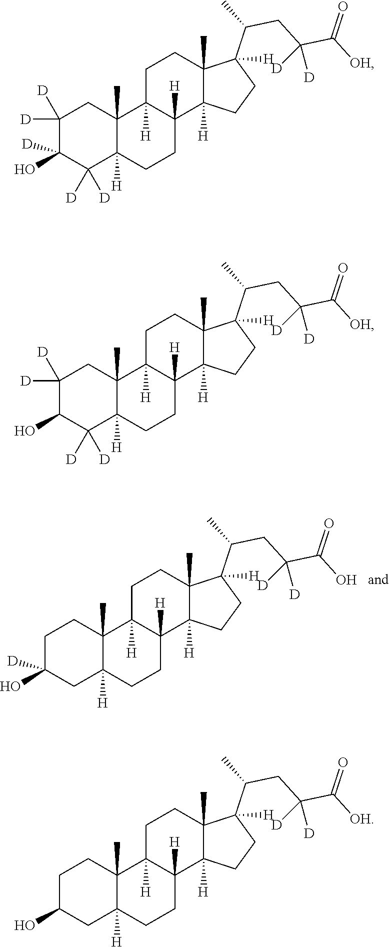

In an even more preferred embodiment, the compound of Formula (II) or a pharmaceutically acceptable salt, co-crystal or solvate thereof is selected from

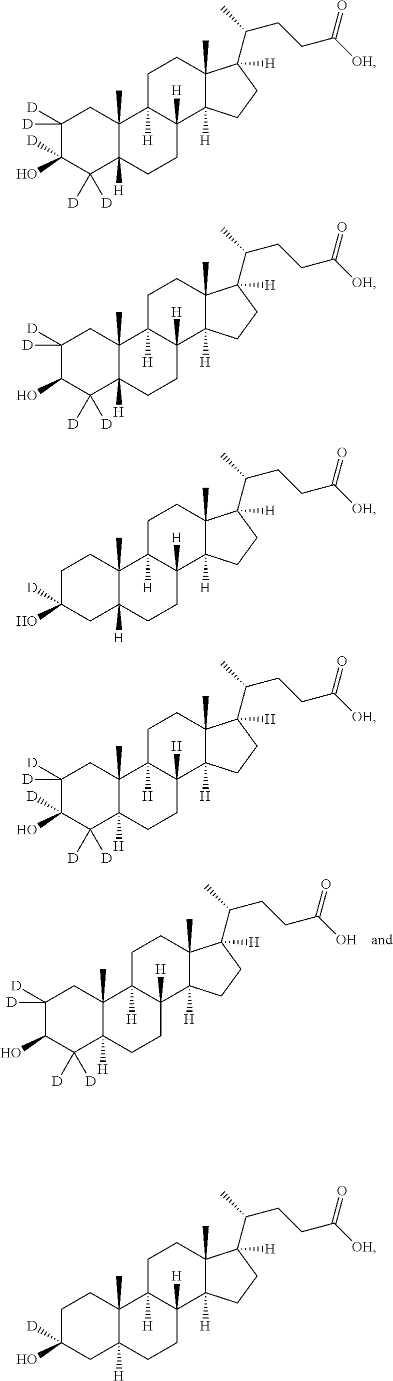

In a most preferred embodiment, the compound of Formula (II) or a pharmaceutically acceptable salt, co-crystal or solvate thereof is

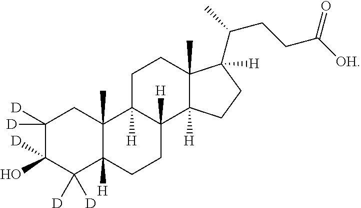

In an equally most preferred embodiment, the compound of Formula (II) or a pharmaceutically acceptable salt, co-crystal or solvate thereof is

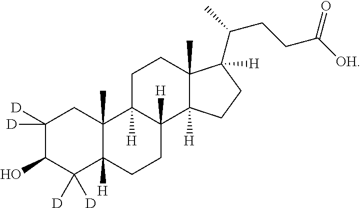

In an equally most preferred embodiment, the compound of Formula (II) or a pharmaceutically acceptable salt, co-crystal or solvate thereof is

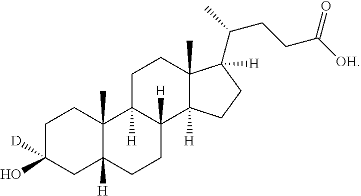

It is to be understood that any of the above mentioned embodiments can be combined with each other in any combination of two or more embodiments.

In a further aspect, the invention relates to the compound according to Formula (II) or a pharmaceutically acceptable salt, co-crystal or solvate thereof for use as a medicament.

In a further aspect, the invention relates to the use of the compounds according to Formula (II) or a pharmaceutically acceptable salt, co-crystal or solvate thereof in preventing or treating a Clostridium difficile-associated disease.

In yet a further aspect, the invention also relates to a pharmaceutical composition comprising a compound according to Formula (I) or Formula (II) or a pharmaceutically acceptable salt, co-crystal or solvate thereof and a pharmaceutically acceptable carrier or excipient.

In yet a further aspect, the invention also relates to a pharmaceutical composition comprising a compound according to Formula (II) or a pharmaceutically acceptable salt, co-crystal or solvate thereof and a pharmaceutically acceptable carrier or excipient.

The disclosure also includes “deuterated analogs” of compounds of Formula (I) and Formula (II) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) and Formula (II) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium (see Experimental Section for details).

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present on these groups and can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Further, the compounds of the present invention may be present in the form of co-crystals. Co-crystals consist of two or more components that form a unique crystalline structure having unique properties. More preferred, co-crystals are solids that are crystalline single phase materials composed of two or more different molecular or ionic compounds generally in a stoichiometric ratio which are neither solvates nor simple salts.

Further, the compounds of the present invention may be present in the form of solvates, such as those which include as solvate water, or pharmaceutically acceptable solvates, such as alcohols, in particular ethanol.

Furthermore, the present invention provides pharmaceutical compositions comprising at least one compound of the present invention, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier.

The compounds described by Formula (I) or Formula (II) are useful for preventing or treating diseases associated with C. difficile. Exposure to C. diff. may lead i.e. in elderly and immune-compromised people to colonization and infection. Once colonized, C. diff, produces toxins which results in a range of clinical signs and symptoms, from inflammation of the mucosal epithelium, diarrhea and cramping in mild cases to the development of pseudomembranous colitis and death in severe cases. Pseudomembranous colitis and C. diff, colitis represent the more severe clinical pictures.

Compositions comprising a compound according to Formula (I) or Formula (II) or a pharmaceutically acceptable salt, co-crystal or solvate thereof are suitable for oral, rectal, topical, parenteral (including subcutaneous, intramuscular and intravenous), ocular (ophthalmic), pulmonary (nasal or buccal inhalation) or nasal administration, although the most suitable route in any given case will depend on the nature and severity of the conditions being treated and on the nature of the active ingredient. If the intended treatment is the treatment of diseases associated with C. difficile, the preferred administration route is the oral or rectal administration. The compositions may be conveniently presented in unit dosage form and prepared by any of the methods well-known in the art of pharmacy.

### Experimental Section

### Abbreviations

EA ethyl acetate
FCC flash column chromatography on silica gel
h hour(s)
PE petroleum ether
sat. saturated (aqueous)

Isolithocholic acid (CAS: 1534-35-6) is commercially available (e.g. Steraloids catalogue ID: C1475-000) or can be prepared from LCA by Mitsunobu reaction using 4-nitrobenzoic acid, triphenylphosphine and diethyl diazodicarboxylate and subsequent saponification with aqueous KOH (P. Miro et al. Chem. Commun. 2016; 52:713).

In an alternative approach, the same procedure can be applied starting with LCA methyl ester:

### Example 1

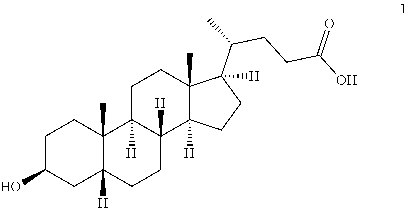

### Step 1: (3S,5R,8R,9S,10S,13R,14S,17R)-17-((R)-5-Methoxy-5-oxopentan-2-yl)-10,13-dimethylhexadecahydro-1H-cyclopenta[a]phenanthren-3-yl 4-nitrobenzoate (1a)

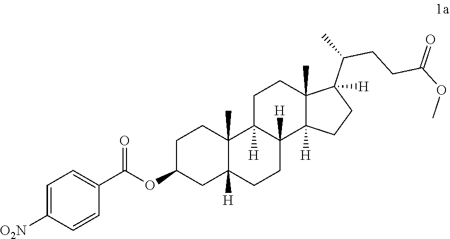

To a solution of lithocholic acid methyl ester (19.5 g, 50 mmol), 4-nitrobenzoic acid (8.4 g, 50 mmol) and PPh₃ (13.1 g, 50 mmol) in tetrahydrofuran (500 mL) was added diisopropyl azodicarboxylate (75 mL, 75 mmol) under N₂ at 0° C. and then the mixture was stirred at rt overnight. The mixture was filtered and the solid was dried under reduced pressure to afford the compound 1a as a white solid.

### Step 2: Isolithocholic Acid (1)

To a solution of compound 1a (17.6 g, 33 mmol) in tetrahydrofuran (300 mL) was added 1N NaOH (50 mL) and then the mixture was stirred at rt for 1 h, quenched by addition of 1N HCl (60 mL) and extracted with EA (3×300 mL). The combined organic layer was concentrated and purified by FCC (EA:PE=3:1) to give compound 1 as a white solid. ¹H-NMR (500 MHz, DMSO-d6): δ 11.95 (br s, 1H), 4.17 (br s, 1H), 3.87 (s, 1H), 2.27-2.21 (m, 1H), 2.13-2.04 (m, 1H), 1.93-1.62 (m, 6H), 1.54-1.48 (m, 1H), 1.40-0.98 (m, 19H), 0.89 (s, 3H), 0.87 (d, J=6.5 Hz, 3H), 0.61 (s, 3H), MS found: 375.2 [M−1]⁻.

### Example 2

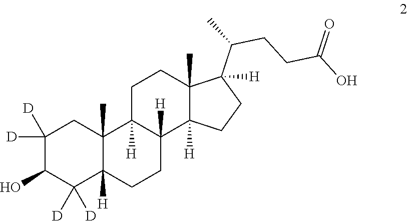

### Step 1: 2,2,4,4-d4-Lithocholic Acid Methyl Ester (2a)

To a solution of 2,2,4,4-d4-lithocholic acid (480 mg, 1.3 mmol) in MeOH (50 mL) was added para-toluenesulfonic acid (20 mg, 0.10 mmol) and then the mixture was stirred at rt overnight. The mixture was quenched by 5% aq. NaHCO₃ and extracted with EA (3×100 mL). The combined organic layer was concentrated to give compound 2a as a white solid.

### Step 2: 2,2,4,4-d4-Isolithocholic Acid (2)

Compound 2a was treated as described in Example 1 to afford compound 2 as a white solid. MS found: 379.3 [M−1]⁻.

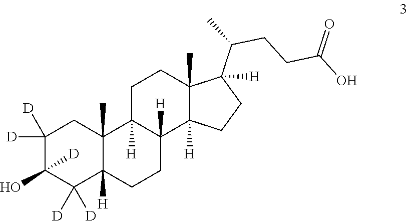

### Example 3

### 2,2,3,4,4-d5-Isolithocholic Acid (3)

2,2,3,4,4-d5-Lithocholic acid was treated as described in Example 1 and Example 2 to afford compound 3 as a white solid. MS found: 380.3 [M−1]⁻.

### Example 4

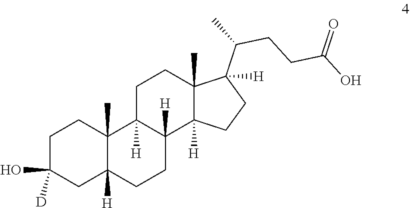

### 3-d1-Isolithocholic Acid (4)

To a solution of 3-oxo-5β-cholanoic acid (5.00 g, 13.4 mmol) in CD₃OD (30 mL) was added NaBD₄ (730 mg, 17.4 mmol) at 0° C. The mixture was stirred at this temperature for 3 h, quenched with sat. NH₄Cl (100 mL) and extracted with EA (3×200 mL). The combined organic layer was concentrated and purified by FCC (EA:PE=3:1) to afford compound 4 as a white solid (beside the 3α-hydroxy isomer). ¹H-NMR (500 MHz, DMSO-d6) δ: 11.93 (s, 1H), 4.13 (s, 1H), 2.25-2.18 (m, 1H), 2.14-2.06 (m, 1H), 1.93-1.62 (m, 6H), 1.55-0.96 (m, 20H), 0.89 (s, 3H), 0.87 (d, J=6.5 Hz, 3H), 0.61 (s, 3H). MS found: 376.3 [M−1]⁻.

### Example 5

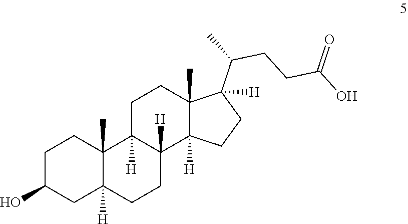

### 3β-Hydroxy-5α-cholan-24-oic Acid (5)

3β-Hydroxy-5α-cholan-24-oic acid (isoallolithocholic acid) is commercially available, e.g. from Steraloids (catalog ID: 00700-000).

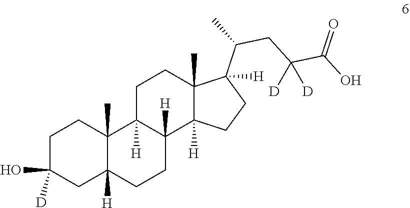

### Example 6

### 3,23,23-d3-Isolithocholic Acid (6)

If one were to treat the methyl ester of Example 4 with LiO^{t}Bu in MeOD at 80° C. under microwave irradiation followed by saponification in NaOD/D₂O in MeOD at 60° C. for 30 min (J. Label. Compd. Radiopharm. 2018; 61:799) one would obtain Example 6.

### Example 6-1 to 6-4

If one were to use a similar procedure as described for Example 6, the following compounds can be prepared:

| # | educt | structure |
|---|---|---|
| 6-1 | Example 1 (iso- LCA) | 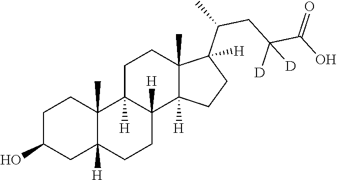 |
| 6-2 | Example 2 | 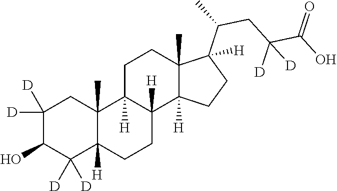 |
| 6-3 | Example 3 | 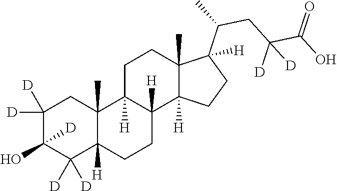 |
| 6-4 | Example 5 (isoallo- LCA) | 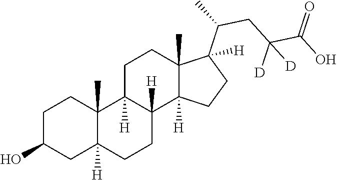 |

### Example 200

Determination of Minimal Inhibitory Concentrations (MICs) of Bile Acid Derivatives on Clostridium difficile (Clostridioides difficile), Human Strain R20291, Ribotype RT027, Reference DSMZ (DSM-27147)

All bacterial culturing steps and MIC experiments were performed under anoxic conditions (95% N₂, 5% H₂) and 37° C. in an incubator model 2002 that was placed in a Type B vinyl anaerobic chamber, both from Coy Laboratories Products. The strains were maintained as frozen stock cultures in Brain-Heart Infusion broth supplemented with 5% (w/v) yeast extract and 1% (w/v) L-cysteine (BHIS) containing 40% (v/v) glycerine (Carl Roth GmbH, Cat. #3783.1) at −80° C. Brain-heart infusion broth (Sigma Aldrich, Cat. #53286) with the addition of with 5 g/L yeast extract (Carl Roth GmbH, Cat. #2363.5) was prepared according to the manufacturer's instructions. After heat sterilization, 1 g L-cysteine (Sigma Aldrich, Cat. #C7352) was added that was dissolved in 10 mL of distilled water (H₂O_{dd}) and filter sterilized. Medium was placed over night into the anaerobic chamber for degassing. For the MIC-assay cryo-preserved strains were streaked out on BBL™ Columbia CNA Agar with 5% Sheep Blood (BD™, Cat. #221352) and grown for 2-3 d. These plates were kept at rt in the anaerobic chamber for up to two weeks for starting fresh liquid cultures. Several colonies were picked for inoculating 5 mL of BHIS in 50 mL tubes (Sarstedt, Cat. #62.547.254) and bacteria were grown overnight. Hundred microliters of this culture were used for inoculating 10 mL of fresh BHIS in a 50 mL tube and bacteria were grown to an optical density at 600 nm (OD₆₀₀) of 0.6-0.8. The inhibitory action of the bile acid derivatives on C. diff. strains were performed in a 96-well format. For this, a 1:1 a dilution series in the range of 2 mM to 8 μM was prepared for each compound in 100 μL BHIS containing 10% (v/v) DMSO (Sigma Aldrich, Cat. #D8418) per well. The bacterial culture was diluted to an OD₆₀₀ of 0.1 in 15 mL BHIS and 100 μL were transferred into each well of the dilution series, resulting in compound concentrations ranging from 1 mM to 4 μM and a final DMSO concentration of 5% (v/v). As a control, bacteria were grown in BHIS with 5% (v/v) DMSO. After 16 h of incubation, bacterial growth was monitored by measuring OD₆₀₀ in a Varioskan microplate reader (ThermoFisher Scientific).

Results: The MIC for Example 1 was measured to be 16 μM and the MIC for Example 5 was measured to be 8 μM for the RT027 ribotype.

### Example 201

Determination of Minimal Inhibitory Concentrations (MICs) of Bile Acid Derivatives on Clostridium difficile (Clostridioides difficile), Mouse Strain VPI 10463 (ATCC 43255)

Concentrations (0.015-250 μM) of test compounds are prepared by serial two-fold dilutions in pre-reduced brain heart infusion (BHI) broth. To each well containing test article, approximately 5×105 CFU of bacteria are added and incubated for 48 hours in an anaerobic chamber at 37° C. Following incubation, the MIC of each test article is determined by presence/absence of bacterial growth in each well.

Results: The MIC for Example 1 and Example 4 was measured to be 15.6 μM, while the MIC for Example 2 and Example 3 was measured to be between 7.8 to 15.6 μM and the MIC for Example 5 was measured to be 3.9 μM for VPI 10463.

### Example 202

One-Hybrid Reporter Assay for the Vitamin D Receptor

The vitamin D receptor (VDR; NR1I1) reporter assay was performed by transient co-transfection of HEK293 cells with pCMV-BD (Stratagene #211342) containing the GAL4 DNA-binding domain fused with the ligand binding domain of VDR (Genbank accession no. NP_000378, aa 88-427), pFR-Luc reporter and pRL-CMV reporter (Promega #E2261) using PEI solution (Sigma Aldrich cat #40872-7) in a 96-well plate. Cells were incubated for 4-6 hours, and then cultured in MEM supplemented with 8.7% FCS, Glutamax, NEAA, sodium pyruvate and Pen/Strep in the presence of test compounds for 16-20 hours. Cells were incubated for 4 to 6 hours in 30 μL/well transfection mix in OPTIMEM and then cultured for further 16 to 20 hours after addition of 120 μL MEM supplemented with 8.7% FCS, Glutamax, NEAA, sodium pyruvate and Pen/Strep in the presence of test compounds. Medium was removed and cells were lysed with 1× Passive Lysis Buffer (Promega). Firefly luciferase buffer was then added and firefly luciferase luminescence was read on BMG LUMIstar OMEGA luminescence plate reader. One second later, renilla luciferase buffer was added and renilla luciferase luminescence was read to evaluate cell viability and to be able to normalize for well to well differences in transfection efficiency.

| Materials | Company | Cat. No. |
|---|---|---|
| HEK293 cells | DSMZ | ACC305 |
| MEM | Sigma-Aldrich | M2279 |
| FCS | Sigma-Aldrich | F7542 |
| Glutamax | Invitrogen | 35050038 |
| Pen/Strep | Sigma Aldrich | P4333 |
| Sodium pyruvate | Sigma Aldrich | S8636 |
| Non-essential amino acids (NEAA) | Sigma Aldrich | M7145 |
| PEI | Sigma Aldrich | 40.872-7 |
| Passive lysis buffer (5×) | Promega | E1941 |
| D-Luciferine | PJK | 260150 |
| Coelentrazine | PJK | 260350 |

Results: The AC₅₀ for Example 1 to Example 5 was measured to be inactive in this assay. For comparison, the AC₅₀ for LCA was measured in the range from 19 to 29 μM.

### Example 203

Efficacy Evaluation in a Murine Model of Clostridium difficile-Associated Disease: Acute Model

The efficacy of compounds in suppressing C. diff infection was assessed in C57BL6 female mice. Mice were made vulnerable to C. diff. infection by administration of a cocktail of antibiotics (1% glucose, kanamycin (0.5 mg/mL), gentamicin (44 μg/mL), colistin (1062.5 U/mL), metronidazole (269 μg/mL), ciprofloxacin (156 μg/mL), ampicillin (100 μg/mL) and vancomycin (56 μg/mL)) in drinking water for a period of 9 days. 3 days prior to C. diff. infection, mice received a single dose of clindamycin (10 mg/kg) in a volume of 0.5 mL by oral gavage. After this antibiotic pre-treatment, mice received a challenge of approximately 4.5 log 10 viable spores of strain VPI 10463 (ATCC-43255) administered by oral gavage. Test compounds (bile acid) and placebo were administered in the chow or via oral gavage bid from day −2 through day 4 or 10 or 11. Gavage medium was aqueous, PBS-buffered 0.5% hydroxypropyl methylcellulose (HPMC) suspension. Efficacy of test articles was assessed by enumeration of survival of test animals over 12/15 days following C. diff, challenge and by comparison of mortality, disease severity scores and assessment of body weight against placebo treatment.

Results studies 1, 2 and 3 (using compound Example 1, dosed with 100 mg/kg daily dose via food or via gavage bid):

Survival

| | | | | Total | | % |
|---|---|---|---|---|---|---|
| Study | n | Treatment | Duration | Deaths | Survived | Death |
| 1 | 10 | Placebo | Day −2 to Day 10 | 4 | 6 | 40 |
| 1 | 10 | 0.1% Ex. | Day −2 to Day 10 | 0 | 10 | 0 |
| | | #1 in chow | | | | |
| 2 | 10 | Placebo | Day −2 to Day 10 | 3 | 6 | 30 |
| 2 | 10 | 0.1% Ex | Day −2 to Day 10 | 0 | 10 | 0 |
| | | #1 in chow | | | | |
| 3 | 10 | Placebo | Day −2 to Day 11 | 8 | 2 | 80 |
| 3 | 10 | 0.1% Ex | Day −2 to Day 4 | 2 | 8 | 20 |
| | | #1 in chow | | | | |
| 3 | 10 | Ex. #1 as | Day −2 to Day 4 | 0 | 10 | 0 |
| | | gavage | | | | |

Body Weight

| | | | | Body | |
|---|---|---|---|---|---|
| | | | | weight | SD body |
| Study | n | Treatment | Duration | day 12 | weight |
| 1 | 10 | Placebo | Day −2 to Day 10 | 15.6 | 0.90 |
| 1 | 10 | 0.1% Ex. #1 in chow | Day −2 to Day 10 | 19.0 | 0.97 |
| 2 | 10 | Placebo | Day −2 to Day 10 | 18.3 | 2.96 |
| 2 | 10 | 0.1% Ex. #1 in chow | Day −2 to Day 10 | 20.0 | 1.11 |
| 3 | 10 | Placebo | Day −2 to Day 11 | 16.7 | 2.19 |
| 3 | 10 | 0.1% Ex. #1 in chow | Day −2 to Day 4 | 18.1 | 2.64 |
| 3 | 10 | Ex. #1 as gavage | Day −2 to Day 4 | 15.5 | 1.06 |

Clinical Signs

| | | | % mouse | % mouse |
|---|---|---|---|---|
| | | | days | days |
| | | | with total | with total |
| | | | clinical | clinical |
| Study | Treatment | Duration | score >2 | score >0 |
| 1 | Placebo | Day −2 to Day 10 | 31 | 58 |
| 1 | 0.1% Ex. #1 in chow | Day −2 to Day 10 | 0 | 30 |
| 2 | Placebo | Day −2 to Day 10 | 21 | 66 |
| 2 | 0.1% Ex. #1 in chow | Day −2 to Day 10 | 0 | 65 |
| 3 | Placebo | Day −2 to Day 10 | 63 | 90 |
| 3 | 0.1% Ex. #1 in chow | Day −2 to Day 10 | 15 | 72 |
| 3 | Ex. #1 as gavage | Day −2 to Day 10 | 2 | 30 |

Score 0

Normal: 0

Lethargic: 1

Lethargic+Hunched: 2

Lethargic+Hunched+Wet tail/abdomen: 3

Lethargic+Hunched+Wet tail/abdomen+Hypothermic: 4

The benefit of compound Example 1 could be demonstrated in an acute mouse model of C. diff, infection. Whereas in the vehicle group 40%, 30% and 80% of the animals died, 100%, 100% and 80% of the animals in the treatment groups survived with improved clinical signs and body weight.

Recurrence Model Mouse

The efficacy of compounds in suppressing recurrent C. diff. infection was assessed in C57BL/6 female mice. Mice were made vulnerable to C. diff. infection by administration of a cocktail of antibiotics (1% glucose, kanamycin (0.5 mg/mL), gentamicin (44 μg/mL), colistin (1062.5 U/mL), metronidazole (269 μg/mL), ciprofloxacin (156 μg/mL), ampicillin (100 μg/mL) and vancomycin (56 μg/mL)) in drinking water for a period of 9 days. 3 days prior to C. diff. infection, mice received a single dose of clindamycin (10 mg/kg) in a volume of 0.5 mL by oral gavage. After this antibiotic pre-treatment, mice received a challenge of approximately 4.5 log 10 viable spores of strain VPI 10463 (ATCC-43255) administered by oral gavage (day 0). After that mice received 50 mg/kg vancomycin from day 0 until day 4. Test articles (bile acid) and placebo were administered via oral gavage bid from day 5 through day 11. Efficacy of test articles was assessed by enumeration of survival of test animals over 15 days following C. diff, challenge and by comparison of mortality, disease severity scores and assessment of body weight against placebo treatment.

Results studies 1 and 2 (using compound Example 1, dosed with 100 mg/kg daily dose via food or via gavage bid):

Survival

| | | | | Total | | % |
|---|---|---|---|---|---|---|
| Study | n | Treatment | Duration | Deaths | Survived | Death |
| 1 | 10 | Placebo | Day −2 to Day 11 | 8 | 2 | 80 |
| 1 | 10 | 0.1% Ex. | Day 5 to Day 11 | 0 | 10 | 0 |
| | | #1 in chow | | | | |
| 2 | 10 | Placebo | Day −2 to Day 11 | 7 | 3 | 70 |
| 2 | 10 | Ex. #1 as | Day 5 to Day 11 | 0 | 10 | 0 |
| | | gavage | | | | |

Body Weight

| | | | | Body weight | SD body |
|---|---|---|---|---|---|
| Study | n | Treatment | Duration | day 12 | weight |
| 1 | 10 | Placebo | Day −2 to Day 10 | 16.7 | 2.19 |
| 1 | 10 | 0.1% Ex. #1 | Day −2 to Day 10 | 18.4 | 0.86 |
| | | in chow | | | |
| 2 | 10 | Placebo | Day −2 to Day 11 | 17.3 | 1.50 |
| 2 | 10 | Ex. #1 as gavage | Day 5 to Day 11 | 16.9 | 1.28 |

Clinical Signs

| | | | % mouse | % mouse |
|---|---|---|---|---|
| | | | days | days |
| | | | with total | with total |
| | | | clinical | clinical |
| Study | Treatment | Duration | score >2 | score >0 |
| 1 | Placebo | Day −2 to Day 10 | 63 | 90 |
| 2 | 0.1% Ex. #1 | Day −2 to Day 10 | 1 | 30 |
| | in chow | | | |
| 2 | Placebo | Day −2 to Day 11 | 38 | 63 |
| 2 | Ex. #1 as gavage | Day 5 to Day 11 | 0 | 56 |

Score 0

Normal: 0

Lethargic: 1

Lethargic+Hunched: 2

Lethargic+Hunched+Wet tail/abdomen: 3

Lethargic+Hunched+Wet tail/abdomen+Hypothermic: 4

The benefit of compound Example 1 could be demonstrated in a recurrence mouse model of C. diff. infection. Whereas in the vehicle group 70 to 80% of the animals died, none of the animals in the treatment groups died with improved clinical signs and body weights.

Example 3 (30 mg/kg) was tested in the same mouse recurrence model against Example 1 (100 mg/kg) and surprisingly, even at lower doses, the effect of the deuteration on the efficacy could be demonstrated. Example 3 performed better as Example 1 with survival of 60% of the animals as compared to 30%.

The comparison of Examples 1 and Example 3 in the mouse recurrence model is shown in FIG. 3.

Recurrence Model Hamster

The efficacy of compounds in suppressing recurrent C. diff infection was assessed in male Syrian hamster. Hamster were made vulnerable to C. diff. infection by administration 1 day prior to C. diff, infection of a single dose of clindamycin (30 mg/kg) by oral gavage. After this antibiotic pre-treatment, mice received a challenge of approximately 1560 viable spores of strain BI1 administered by oral gavage (day 0). After that hamster received 10 mg/kg vancomycin from day 0 until day 5. Test articles (bile acid) and placebo were administered via oral gavage bid from day 6 through day 15. Efficacy of test articles was assessed by enumeration of survival of test animals over 20 days following C. diff. challenge and by comparison of mortality, disease severity scores and assessment of body weight against placebo treatment.

The benefit of compound Example 1 could be demonstrated in a recurrence hamster model of C. diff. infection. Whereas in the vehicle group 20% of the animals died, none of the animals in the treatment group died with improved clinical signs and body weights.

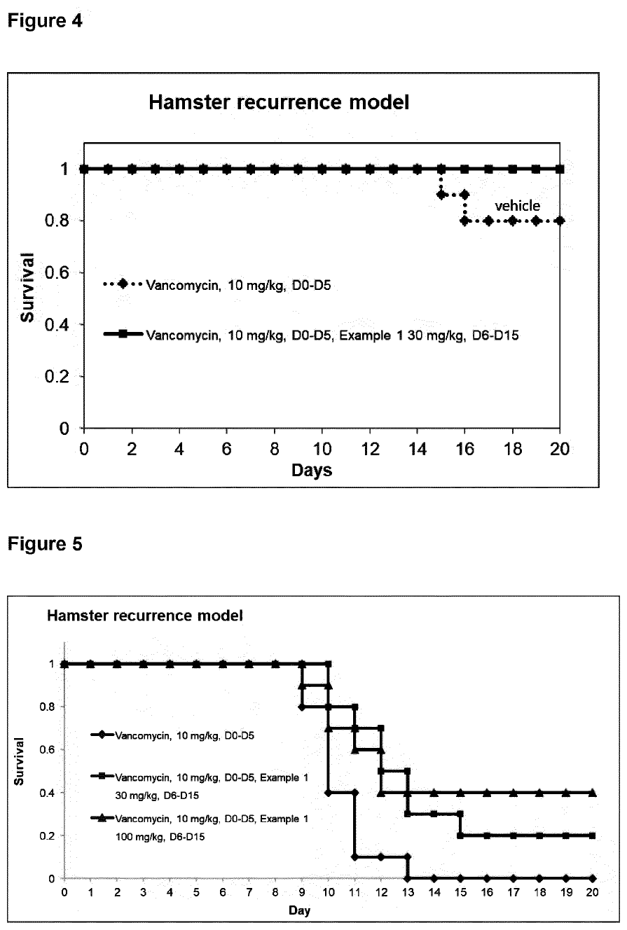

The results are illustrated in FIG. 4.

Since the mortality in the vehicle group with 20% dead animals was quite low, the experiment was repeated at the same facility. In the new experiment, 100% of the vehicle animals died within 13 days. Again, the benefit of compound Example 1 could be demonstrated in the recurrence hamster model of C. diff. infection. A dose dependent efficacy was demonstrated with 20% survivors at 30 mg/kg and 40% survivors at 100 mg/kg (p-values: 0.0074 and 0.0048, respectively).

The results are illustrated in FIG. 5.

### Summary

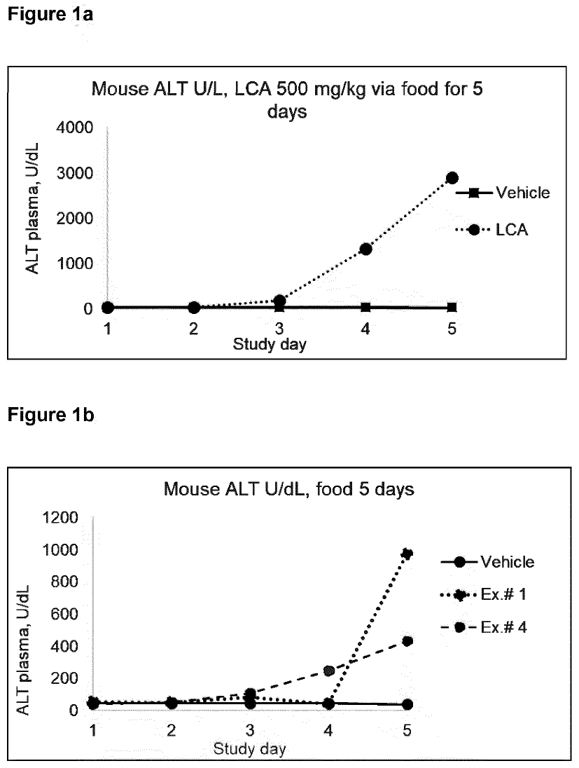

Lithocholic acid (LCA) is claimed in WO2010/062369 (Claim 9) to be useful in preventing C. diff.-associated diseases but no in vitro and in vivo data for this assumption was presented. Even more, for closest analog in regard to our invention, 3-acetyl-LCA (Table 3, line 9 in WO2010/062369) the underlying mechanism (i.e. inhibition of germination) was reported to be not present. On the other hand, for LCA several liabilities in the literature are reported: for example oral administration of LCA results in elevation of alanine transaminase (ALT) indicating hepatocellular injury. We were able to confirm this described liability of ALT-elevation at even lower dose (FIG. 1a). As shown in FIG. 1b (different y-axis scaling compared to FIG. 1a), this liability of ALT-elevation is less pronounced for iso-LCA (Example 1) and even lesser pronounced for a deuterated iso-LCA analog (Example 4). Another potential liability of LCA is increased Vitamin D agonism leading to hypercalcemia followed by polyuria. We confirmed this Vitamin D agonism for LCA and showed, that iso-LCA and analogs are devoid of this Vitamin D agonism (Example 202).

Although it could be argued, that modifying LCA by simply isomerizing the 3-hydroxy position towards iso-LCA (and also in the allolithocholic acid case, i.e. 5α-analogs) could be considered as trivial or obvious, we surprisingly found that this structurally minor modification (on plain paper) shows additional unexpected beneficial effects:
still similar active or even more potent compared to LCA in the in vitro assays measuring the minimal inhibitory concentrations on growth of C. diff in different strains (Example 200/201)
the other secondary bile acid iso-DCA showed to be inactive in the in vitro assay measuring the MIC on growth of C. diff. in the mouse strain (Example 201) at concentrations up to 250 μM

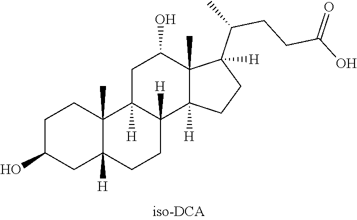

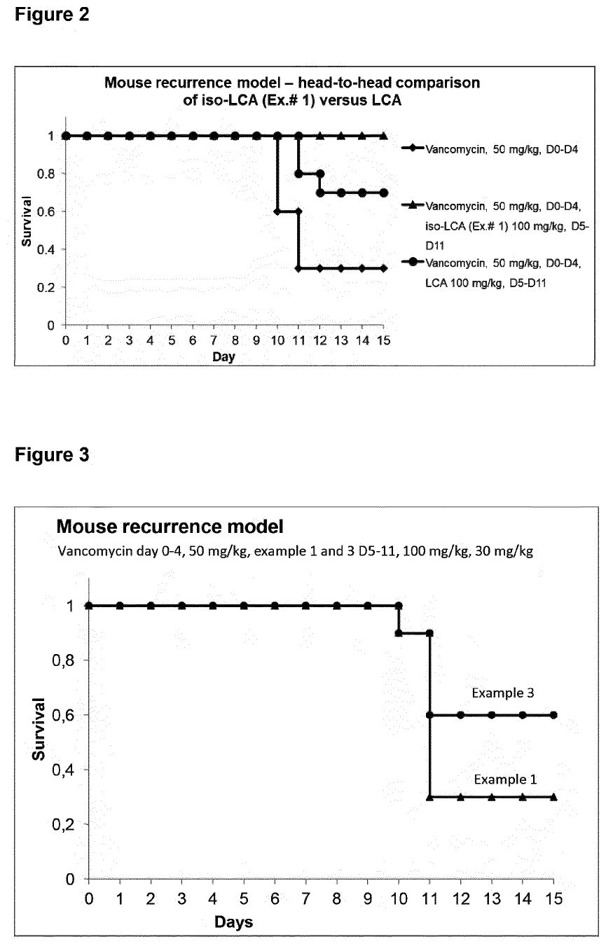

as mentioned above, contrary to LCA no Vitamin D agonism was measurable for the iso-LCA analogs (Example 202)
in a head-to-head comparison of iso-LCA and LCA we showed a better efficacy expressed as a higher surviving rate in the recurrence mouse model (Example 202; FIG. 2)
in additional animal models, iso-LCA and analogs show beneficial effect (i.e. high surviving rate) and a deuterated analog of iso-LCA was even more beneficial in a mouse model compared to non-deuterated iso-LCA (FIG. 3)
Iso-LCA (Example 1) was tested in a recurrence hamster study of C. diff. infection at Evotec (UK): The compound was protective to 20-40% of the animals in a dose-dependent manner (FIG. 5). Treatment with Example 1 removed all spores from caecum and colon in surviving animals. The results are comparable to the published results obtained with the toxin B antibody bezlotoxumab (P. Warn et al. Antimicrob. Agents Chemother. 2016; 60:6471).
A further surprising improvement was the deuteration of iso-LCA with enhanced in vivo efficacy and reduced liver toxicity. The deuterium labelling, especially at the 3-position of the bile acid, presumably leads to a decreased epimerization of iso-LCA to LCA and is therefore less toxic and more efficacious.

What is claimed:

1. A compound according to Formula (I) 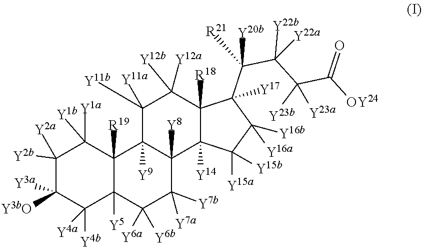
or a pharmaceutically acceptable salt, co-crystal or solvate thereof,
wherein
each R¹⁸, R¹⁹ and R²¹ is independently selected from —CH₃, —CH₂D, —CHD₂ and —CD₃; and
each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium,
for use in preventing or treating a Clostridium difficile-associated disease in a mammalian subject.

2. The compound according to Formula (I) or a pharmaceutically acceptable salt, co-crystal or solvate thereof for use according to claim 1, which is selected from

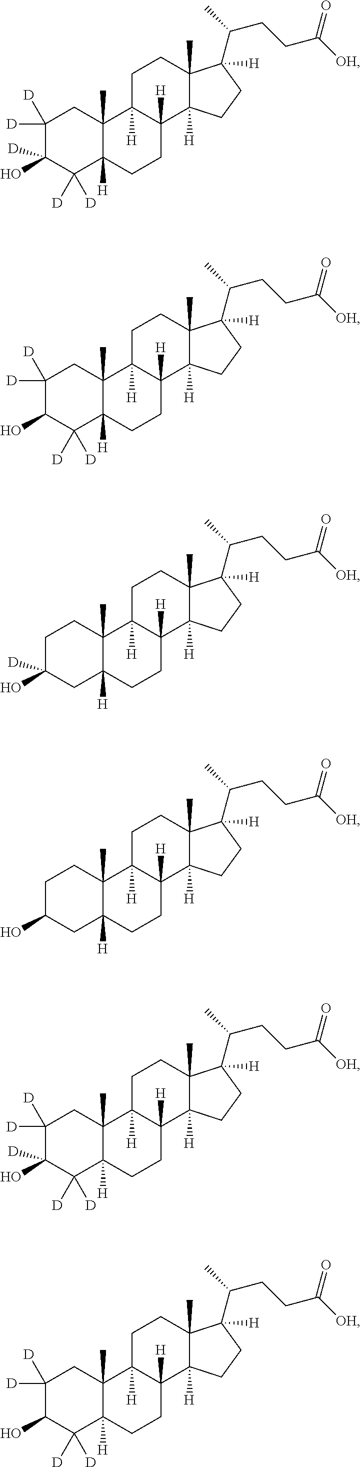

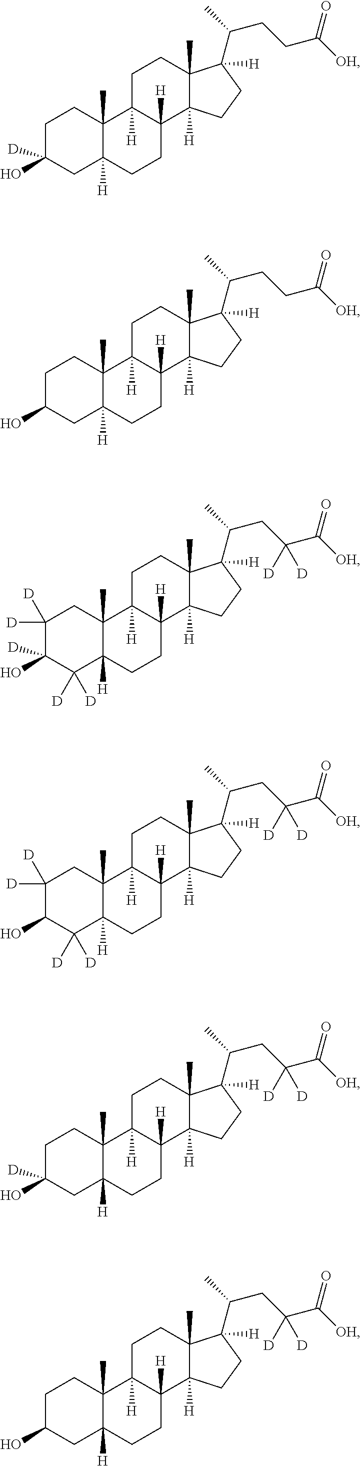

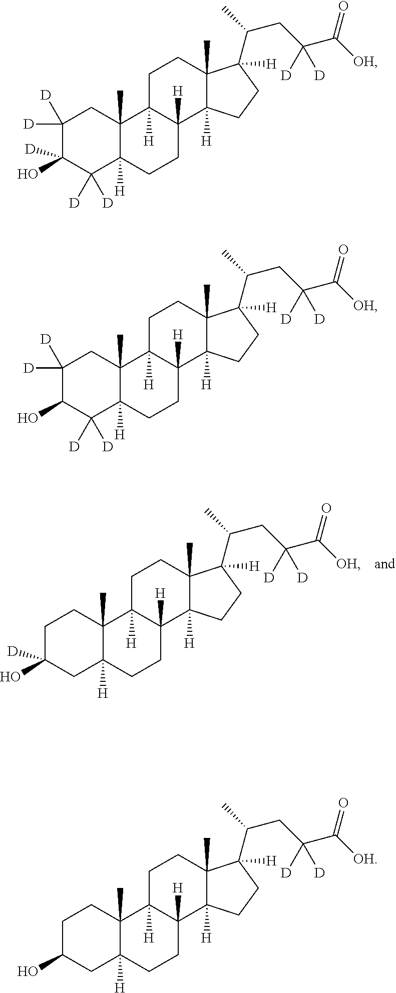

3. The compound according to Formula (I) or a pharmaceutically acceptable salt, co-crystal or solvate thereof for use according to claim 1, wherein Y⁵ is in the beta-orientation.

4. The compound according to Formula (I) or a pharmaceutically acceptable salt, co-crystal or solvate thereof for use according to anyone of claims 1 to 3, which is selected from

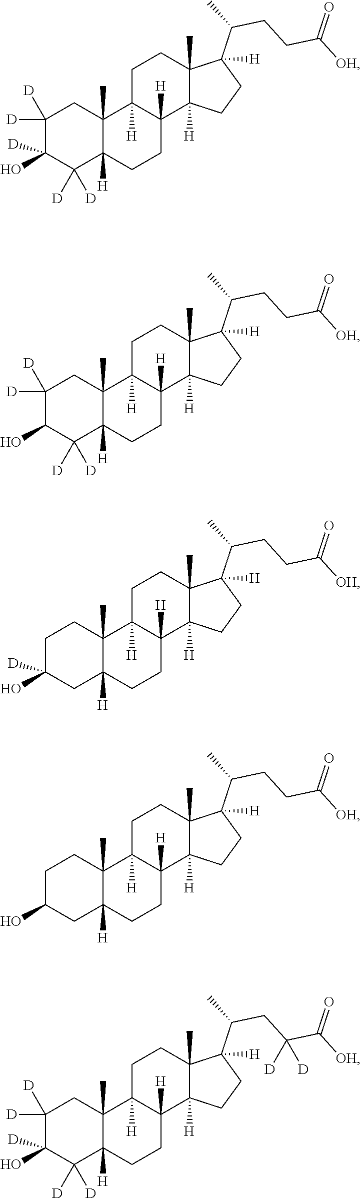

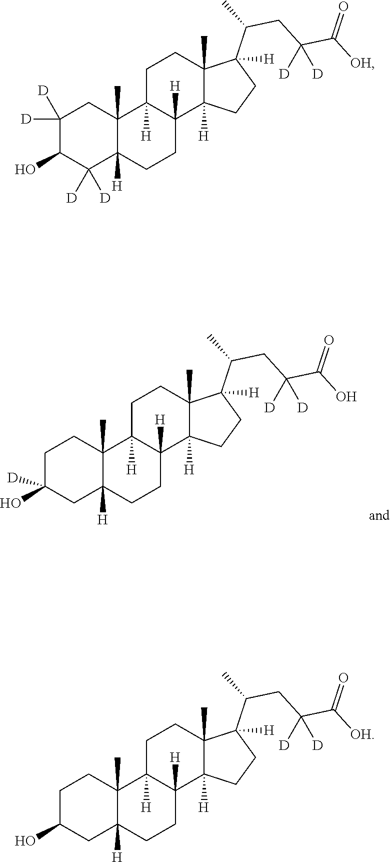

5. The compound according to Formula (I) or a pharmaceutically acceptable salt, co-crystal or solvate thereof for use according to claim 1 or 2, which is

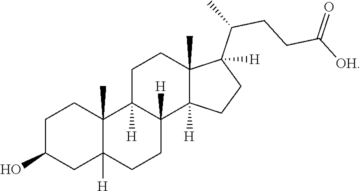

6. The compound according to Formula (I) or a pharmaceutically acceptable salt, co-crystal or solvate thereof for use according to claim 5, which is

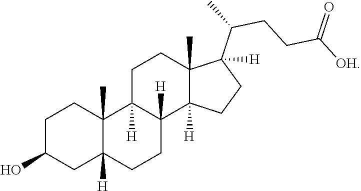

7. The compound according to Formula (I) or a pharmaceutically acceptable salt, co-crystal or solvate thereof for use according to claim 1 or 2, which is

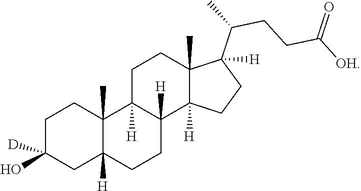

8. A compound represented by Formula (II) 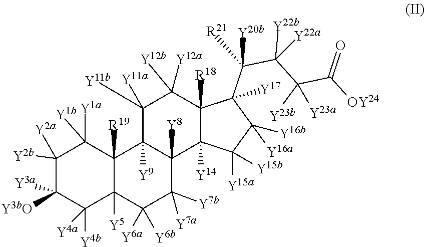
or a pharmaceutically acceptable salt, co-crystal or solvate thereof, wherein
each R¹⁸, R¹⁹ and R²¹ is independently selected from —CH₃, —CH₂D, —CHD₂ and —CD₃;
each Y^{1a}, Y^{1b}, Y^{2a}, Y^{2b}, Y^{3a}, Y^{3b}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, Y^{23b} and Y²⁴ is independently selected from hydrogen or deuterium;
with the proviso that
at least one of Y^{1a}, Y^{1b}, Y^{2a}, Y^{1b}, Y^{3a}, Y^{4a}, Y^{4b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, Y^{23a}, and Y^{23b} is deuterium; or
at least one of R¹⁸, R¹⁹ and R²¹ is selected from —CH₂D, —CHD₂ and —CD₃.

9. The compound of Formula (II) or a pharmaceutically acceptable salt, co-crystal or solvate thereof according to claim 8, wherein
each Y^{2a}, Y^{2b}, Y^{3a}, Y^{3b}, Y^{4a}, Y^{4b}, Y^{23a}, Y^{23b} is independently selected from hydrogen or deuterium;
each R¹⁸, R¹⁹ and R²¹ is —CH₃; and
each Y^{1a}, Y^{1b}, Y⁵, Y^{6a}, Y^{6b}, Y^{7a}, Y^{7b}, Y⁸, Y⁹, Y^{11a}, Y^{11b}, Y^{12a}, Y^{12b}, Y¹⁴, Y^{15a}, Y^{15b}, Y^{16a}, Y^{16b}, Y¹⁷, Y^{20b}, Y^{22a}, Y^{22b}, and Y²⁴ is hydrogen,
with the proviso that
at least one of Y^{2a}, Y^{2b}, Y^{3a}, Y^{3b}, Y^{4a}, Y^{4b}, Y^{23a}, Y^{23b} is deuterium.

10. The compound of Formula (II) or a pharmaceutically acceptable salt, co-crystal or solvate thereof according to claim 8 or 9, which is selected from

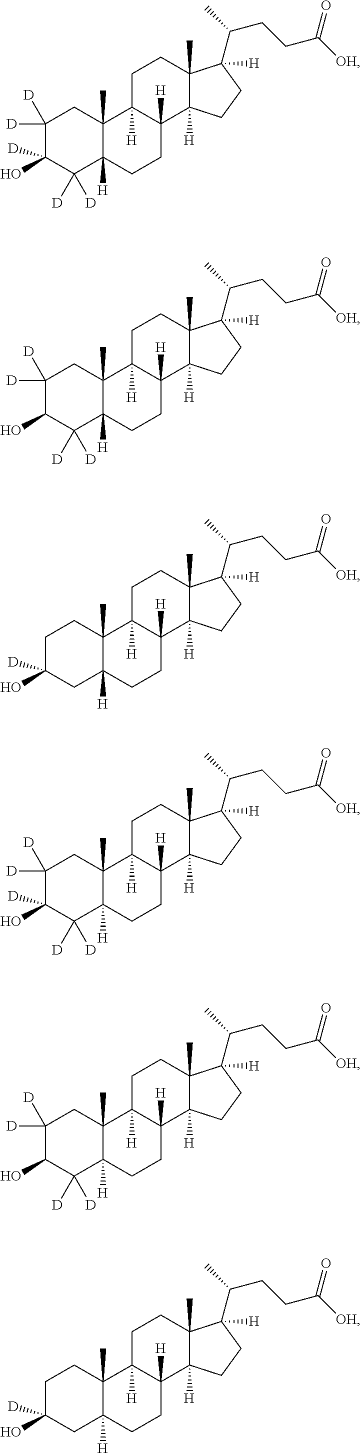

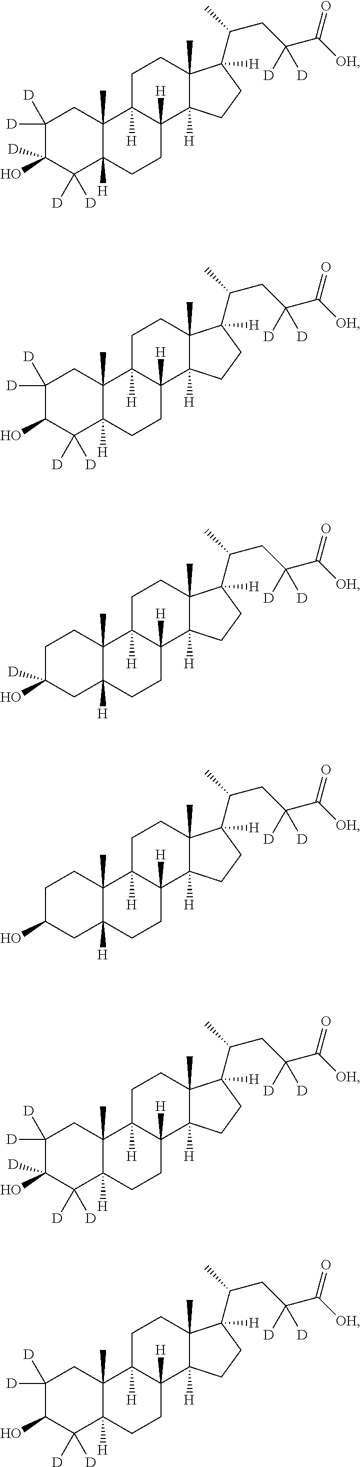

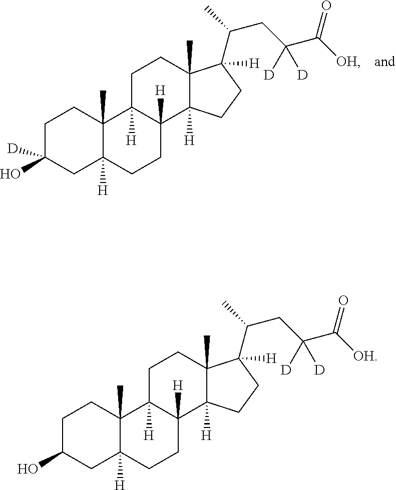

11. The compound of Formula (II) or a pharmaceutically acceptable salt, co-crystal or solvate thereof according to any of claim 8 to 10, which is selected from

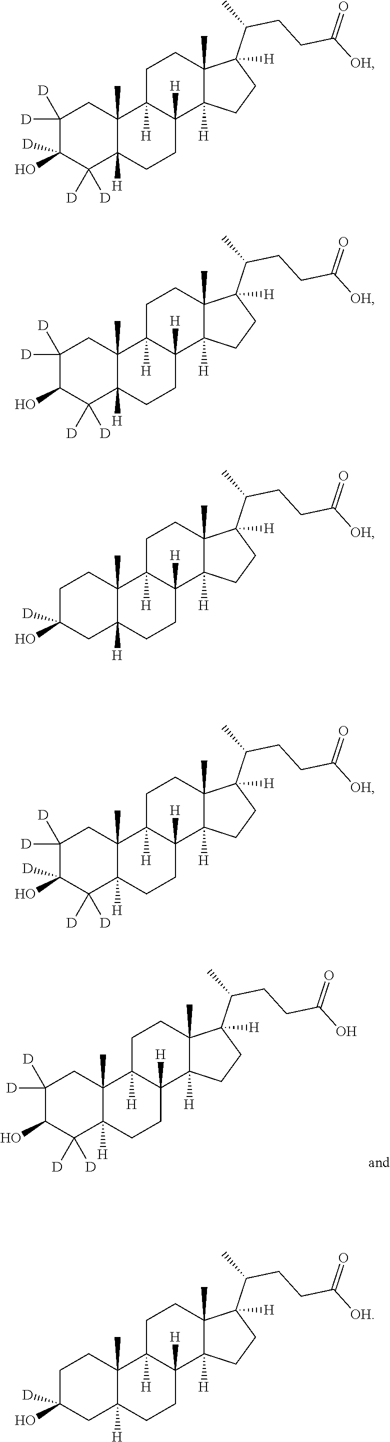

12. A compound of Formula (II) or a pharmaceutically acceptable salt, co-crystal or solvate thereof according to any of claims 8 to 11 for use as a medicament.

13. A compound of Formula (II) or a pharmaceutically acceptable salt, co-crystal or solvate thereof according to any of claims 8 to 11 for use in preventing or treating a Clostridium difficile-associated disease.

14. A pharmaceutical composition comprising a compound of Formula (I) or Formula (II) or a pharmaceutically acceptable salt, co-crystal or solvate thereof according to any of claims 1 to 11 and a pharmaceutically acceptable carrier or excipient.

15. A pharmaceutical composition comprising a compound of Formula (II) or a pharmaceutically acceptable salt, co-crystal or solvate thereof according to any of claims 8 to 11 and a pharmaceutically acceptable carrier or excipient.
